(19) United States
(12) Patent Application Publication
(10) Pub. No.: US 2014/0234860 A1
(43) Pub. Date: Aug. 21, 2014

(54) MEASUREMENT OF PKA FOR CANCER CHARACTERIZATION

(71) Applicant: TRAXXSSON, LLC, St. Louis, MO (US)

(72) Inventors: Robert S. Puskas, Manchester, MO (US); Douglas Held, Glendale, MO (US)

(21) Appl. No.: 14/114,811

(22) PCT Filed: Apr. 18, 2012
(86) PCT No.: PCT/US12/34049

§ 371 Date: Apr. 23, 2014

Related U.S. Application Data
(60) Provisional application No. 61/477,593, filed on Apr. 20, 2011.

(51) Int. Cl. G01N 33/574; C12Q 1/48

(52) U.S. Cl. CPC G01N 33/57488; C12Q 1/485
USPC 435/7.4; 435/15

(57) ABSTRACT

A method for characterizing a carcinoma in a subject. The method comprises assaying a sample of a bodily fluid derived from the subject for extracellular PKA activity and comparing the activity to a reference value. In the assay, a reaction mixture is prepared comprising the previously unfrozen sample, a PKA peptide substrate, a phosphorylation agent, the prepared mixture is incubated, and phosphorylated substrate formed in the incubated mixture is detected. The reference value is the amount of phosphorylated substrate formed in a mixture under equivalent redox conditions for a sample of bodily fluid derived from a population of normal subjects of the same species.

### CROSS-REFERENCE TO RELATED APPLICATIONS

This application claims the benefit of U.S. Provisional Application Ser. No. 61/477,593 filed on Apr. 20, 2011, which is hereby incorporated by reference in its entirety, including any figures, tables, and drawings.

### FIELD OF THE INVENTION

The present invention generally relates to medical diagnostics and to methods, kits and assays for the determination of certain protein biomarkers and the characterization of cancer.

### BACKGROUND OF THE INVENTION

The cAMP-dependent protein kinase (PKA) is activated by the binding of cAMP to the regulatory subunit (R), of the molecule and results in the release of the active catalytic kinase subunit (C). Most of the effects of cAMP in eukaryotic systems are the result of phosphorylation of proteins at serine or threonine residues by PKA. There are several isoforms of both subunits of PKA. PKA is localized subcellularly by binding to multidomain scaffolding proteins known as AKAPs which bind to the R subunits of the holoenzyme [1]. More than 50 AKAPs are known which localize PKA in various cell types. The PKA-specific inhibitor (PKI) acts by binding with high affinity to the substrate binding site of the free active catalytic subunit [2].

As a result of work done at the National Cancer Institute (NCI) and by others, it has been reported that the activity level of PKA is elevated in the plasma and serum of cancer patients, and that anti-PKA antibody levels are elevated in the serum of cancer patients [3-5].

Like most current cancer screening tests, the PSA (prostate-specific antigen) blood test for prostate cancer is of questionable value. In fact the American Cancer Society no longer recommends that men routinely have PSA tests as part of their routine physical examinations [7]. There are several reasons for this lack of support for testing. When prostate cancer is present, the PSA test fails to detect 3 out of 4 cases [8]. In addition, when the PSA test comes back positive, 3 out 4 times it is a false positive—the patient does not have cancer [9]. Nonetheless, patients who have a positive PSA test typically will have a group of 12 or more biopsy samples taken from their prostate to verify if cancer is present. At an average cost of $1,500 per biopsy, the national cost for the 700,000 unnecessary prostate biopsies done each year exceeds $1 billion [10]. Making matters worse, there is a 25% chance that a prostate biopsy will not detect cancer even when cancer is present [11]. Improved patient outcome could be accomplished by replacing a biopsy with a cancer confirmatory blood test, for a savings in health care costs of nearly $900 million annually.

Mammograms have a poorer record than PSA tests. While mammograms are purported to detect 85-90% of breast cancers when they are present, the detected tumors on average are 1½ inches in diameter when diagnosed. As for all cancers, earlier detection leads to better patient outcomes. What makes breast cancer screening costly is that an estimated 95% of the positive mammograms are false positives—the patient does not have cancer [12]. A positive mammogram frequently leads to a breast biopsy. A typical needle biopsy costs about $1,500; an invasive surgical biopsy (about ⅓ of all breast biopsies) costs about $5,000. This brings the national cost for the estimated 2 million unnecessary breast biopsies to more than $5.1 billion annually. Improved patient outcome could be accomplished by substituting a cancer confirmatory test for a biopsy, for a savings in health care costs of almost $5 billion annually

Cancer monitoring tests. Beyond screening tests there are additional blood tests that are used to monitor cancer patients once cancer has been diagnosed. Many of these tests are not specific for cancer or specific for a particular type of cancer, rendering them of limited value as cancer screening tests. However, they can be an effective means for monitoring cancer treatment and testing for disease recurrence. These tests include CA-15.3 and CA27.29 for breast cancer, CA 125 for ovarian cancer, CEA for colon cancer and PSA for prostate cancer [13]. Other blood tests have been used to determine if a primary cancer has spread to other organs. These tests include assays for metastases to bone (osteoprotegrin), and liver (E-selectin).

### SUMMARY OF THE INVENTION

Among the various aspects of the present invention may be noted methods, assays and kits for the measuring the activity of extracellular PKA (“xPKA”) in a fluid sample derived from a bodily fluid of a subject, and the characterization of cancer, and more specifically, carcinoma, in the subject. Without being bound to any particular theory, it has been discovered that extracellular PKA derived from a bodily fluid of a subject afflicted with a carcinoma and extracellular PKA derived from a bodily fluid of a subject not afflicted with a carcinoma can exhibit significantly different activities for the phosphorylation of a PKA substrate and, thus, can be used to characterize carcinoma in a subject.

Briefly, therefore, one aspect of the present invention is directed to a method for characterizing a carcinoma in a subject. The method comprises assaying a sample of a bodily fluid derived from the subject for extracellular PKA activity, the assay comprising preparing a reaction mixture comprising the previously unfrozen sample, a PKA peptide substrate, a phosphorylation agent, incubating the prepared mixture, and detecting phosphorylated substrate formed in the incubated mixture. The amount of phosphorylated substrate formed in the assay is compared to a reference value, the reference value being the amount of phosphorylated substrate formed in a mixture under equivalent conditions for a sample of bodily fluid derived from a population of subjects not afflicted with cancer.

The present invention is further directed to a kit for determining the amount of extracellular PKA activity in a sample. The kit comprises a PKA substrate, a phosphorylation agent, and control PKA of known activity. In one embodiment, the kit further comprises an oxidizing agent. In another embodiment, the kit further comprises a phosphatase inhibitor.

Other objects and features will be in part apparent and in part pointed out hereinafter.

### BRIEF DESCRIPTION OF THE DRAWINGS

FIG. 1 is graph depicting the effect of oxidation reduction potential (mV) upon the ratio of apparent xPKA activity (cancer subjects/normal subjects) as more fully described in Example 1.

FIG. 2 is graph depicting the effect of oxidation reduction potential (mV) upon the ratio of apparent xPKA activity (cancer subjects/normal subjects) as more fully described in Example 2.

FIG. 3 is graph depicting xPKA activity levels for samples with NaF (phosphatase inhibitor) in the reaction mixture as more fully described in Example 3.

FIG. 4 is graph depicting oxidation reduction potential and ratios of Cancer/Normal xPKA activity with NaF (phosphatase inhibitor) in the reaction mixture as more fully described in Example 3.

FIG. 5 is a table presenting apparent xPKA activities in samples from prostate and colon cancer patients relative to those from individuals apparently without cancer with various concentrations of oxidant or reductant used in the sample preparation buffer as more fully described in Example 1.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, “extracellular PKA” or “xPKA” means cAMP-dependent protein kinase A found in bodily fluids outside of bodily cells.

As used herein, “normal subject” or “normal individual” means a subject or individual not known to be afflicted with, or suspected of being afflicted with, a carcinoma.

As used herein, “ORP” means oxidation-reduction potential.

### DETAILED DESCRIPTION OF THE INVENTION

In accordance with one aspect of the present invention, it has been shown that extracellular PKA may be used to characterize carcinoma in a subject. More specifically, it has been determined that extracellular PKA derived from persons unafflicted with carcinoma and extracellular PKA derived from persons afflicted with carcinoma can be differentiated, depending upon the reaction conditions. Under certain reaction conditions, extracellular PKA derived from persons unafflicted with carcinoma has a greater activity for the phosphorylation of a PKA substrate than does extracellular PKA derived from persons afflicted with carcinoma. Under certain other reaction conditions, extracellular PKA derived from persons unafflicted with carcinoma has less activity for the phosphorylation of a PKA substrate than does extracellular PKA derived from persons afflicted with carcinoma. Under yet other reaction conditions, extracellular PKA derived from persons unafflicted with carcinoma and extracellular PKA derived from persons afflicted with carcinoma have approximately equivalent activities for the phosphorylation of a PKA substrate.

We show that depending upon redox conditions apparent xPKA activity in serum of cancer patients can be higher, lower, or the same as that of apparently healthy controls. Thus, apparent xPKA activity may be used as an indicator of the presence of cancer, provided the redox conditions of the assay are known and/or controlled. We demonstrate here redox conditions that achieve each of the above relationships between apparent extracellular PKA activity in cancer patients and normal subjects, and we further define preferred conditions for the use of this assay for detecting cancer.

In accordance with one aspect of the present invention, therefore, the results of an assay for an individual subject under a set of reaction conditions may be compared to a reference value for that set of reaction conditions to characterize carcinoma in that subject. For example, if the assay for the individual subject is carried out under reaction conditions at which the activity of extracellular PKA derived from subjects afflicted with carcinoma for the phosphorylation of a PKA substrate is significantly greater than the activity of extracellular PKA derived from subjects unafflicted with carcinoma for the phosphorylation of a PKA substrate (i.e., normal subjects), and the activity of the individual subject's xPKA for the phosphorylation of PKA substrate is significantly greater than the activity that is characteristic of normal subjects, a diagnosis, prognosis, etc., may be determined. Alternatively, if the assay for the individual subject is carried out under reaction conditions at which the activity of extracellular PKA derived from subjects afflicted with carcinoma for the phosphorylation of a PKA substrate is significantly less than the activity of extracellular PKA derived from normal subjects for the phosphorylation of a PKA substrate, and the activity of the individual subject's xPKA for the phosphorylation of PKA substrate is significantly less than the activity that is characteristic of subjects unafflicted with carcinoma, a diagnosis, prognosis, etc., may be determined.

The relative activities of extracellular PKA derived from subjects afflicted with carcinoma and normal individuals for the phosphorylation of a PKA substrate depends, at least in part, upon the oxidation state of the PKA in the assay. In general, the activity of extracellular PKA from normal individuals appears to be significantly influenced by the redox environment in which it is found. Extracellular PKA from normal individuals appears to have lower activity when the redox environment is oxidizing and higher activity when the environment is highly reducing. Consequently, the apparent activity of extracellular PKA in a fluid sample derived from normal individuals can be increased by treating the sample with a reducing agent to form a mixture that has an ORP value that is more reducing, or decreased by treating the sample with an oxidizing agent. In contrast, the activity of extracellular PKA derived from individuals afflicted with a carcinoma is relatively insensitive to oxidation state. That is, the activity is relatively constant irrespective of whether it is treated with an oxidizing agent or a reducing agent.

In one embodiment, apparent PKA activity is assayed under moderately reducing conditions, i.e., the range of conditions at which the apparent xPKA activity in samples derived from cancer patients is greater than the apparent xPKA activity that is characteristic of normal patients. Moderately reducing conditions may be established, for example, by forming a mixture comprising the sample and a reducing agent wherein the mixture has an ORP value in the range of about −110 mV to about −20 mV. For example, in one embodiment, the mixture containing the sample has an ORP value in the range of about −100 mV to about −90 mV. By way of further example, in one embodiment, the mixture containing the sample has an ORP value in the range of about −20 mV to about −30 mV.

In another embodiment, apparent PKA activity is assayed under oxidizing conditions, or moderately or highly reducing conditions, i.e., the range of conditions at which the apparent xPKA activity in samples derived from cancer patients is less than the apparent xPKA activity that is characteristic of normal patients. Highly reducing conditions may be established, for example, by forming a mixture comprising the sample and a reducing agent wherein the mixture has an ORP value that is less than about −110 mV (that is, conditions that are more reducing than about −110 mV). For example, in one embodiment, the mixture containing the sample has an ORP value of less than about −120 mV. By way of further example, in one embodiment, the mixture containing the sample has an ORP value of less than about −145 mV. Alternatively, moderately reducing or oxidizing conditions may be established, for example, by forming a mixture comprising the sample and an oxidizing agent or a reducing agent wherein the mixture has an ORP value of greater than −20 mV (that is, conditions that are more oxidizing than −20 mV). For example, in one embodiment, the mixture containing the sample has an ORP value of at least about -15 mV. By way of further example, in one embodiment, the mixture containing the sample has an ORP value of at least about 1 mV). By way of further example, in one embodiment, the mixture containing the sample has an ORP value of at least about 60 mV). By way of further example, in one embodiment, the mixture containing the sample has an ORP value of at least about 128 mV.

The sample may be incubated in a mixture having a desired, or at least known redox environment, for a period of time, before the assay is initiated. In certain embodiments, for example, the incubation time will be at least about 1 minute before the PKA activity assay is initiated. Typically, however, greater incubation times will be employed. For example, in one embodiment the incubation time will be at least about 5 minutes. By way of further example, in some embodiments, the incubation time will be at least 10 minutes. By way of further example, in some embodiments, the incubation time will be at least 30 minutes. By way of further example, in some embodiments, the incubation time will be at least about 1 hour. In such embodiments, the incubation temperature may be in the range of 20 to 37° C., with about 25° C. being preferred in certain embodiments. This may be accomplished, for example, in an incubation mixture formed prior to the combination of the sample with the PKA substrate.

Although presently less preferred, in certain embodiments the sample is not incubated with an oxidizing agent or a reducing agent for a period of time before the PKA activity assay is initiated. Rather, a reaction mixture for determining PKA activity is prepared directly from the sample by combining the sample with a PKA peptide substrate, a phosphorylation agent, and optionally a reducing agent or oxidizing agent, the prepared mixture is incubated, and phosphorylated substrate formed in the incubated mixture is detected. In this situation it is generally preferred that the assay be carried out under reaction conditions at which a ratio of the activities for a statistically significant population of normal subjects to a statistically significant population of subjects afflicted with carcinoma be at least 0.05:1 and less than 0.8:1. In certain embodiments, it is preferred that the ratio of the activities for a statistically significant population of normal subjects to a statistically significant population of subjects afflicted with carcinoma be at least 0.075:1 and less than 0.6:1. In certain embodiments, it is preferred that the ratio of the activities for a statistically significant population of normal subjects to a statistically significant population of subjects afflicted with carcinoma be at least 0.1 and less than 0.4:1.

Because relative activities of apparent extracellular PKA from normal subjects and those afflicted with carcinoma depend upon reaction conditions, it is generally preferred that the reaction conditions for an assay be those at which the activities are significantly different. That is, it is generally preferred that the assay be carried out under reaction conditions at which a ratio of the activities for a statistically significant population of normal subjects to a statistically significant population of subjects afflicted with carcinoma be at least 1.2:1 or less than 0.8:1. In certain embodiments, it is preferred that the ratio of the activities for a statistically significant population of normal subjects to a statistically significant population of subjects afflicted with carcinoma be at least 2:1 or less than 0.5:1. In certain embodiments, it is preferred that the ratio of the activities for a statistically significant population of normal subjects to a statistically significant population of subjects afflicted with carcinoma be at least 3:1 or less than 0.2:1.

PKA has two exposed cysteines Cys¹⁹⁹ and Cys³⁴³. It has been determined that sulfhydryl modification of Cys³⁴³ has minimal impact on enzyme activity. However, sulfhydryl modification of Cys¹⁹⁹ predisposes the enzyme to dephosphorylation and inactivation [1]. Humphries et al demonstrated that apparent PKA activity in cell lysates is regulated by an interplay between oxidation of PKA and oxidation of phosphatases. [2] Because these enzymes differentially respond to oxidation, they react differentially to the redox state of the sample, and the overall apparent activity of PKA varies in a complex manner in response to the redox state of a sample.

We have demonstrated in serum that this complex interplay of xPKA and phosphatase activities based on redox state also exists, and that a third regulatory mechanism of xPKA activity may also be observed in blood. When the nonspecific phosphatase inhibitor NaF is added to a reaction mixture comprising serum derived from a normal subject, the apparent xPKA activity is decreased by approximately 50%. In cell lysates when the nonspecific phosphatase inhibitor NaF is added to a PKA reaction mixture, the apparent xPKA activity increases if active phosphatases were present or remains the same if inactive phosphatases were present in the sample. Finding neither of these results, but rather a reduction in xPKA activity in normal serum samples implies that a phosphatase-sensitive regulatory mechanism exists in serum from normal subjects that reduces the activity of xPKA. A corresponding reduction in xPKA activity in response to phosphatase inhibition, however, has not been observed, to-date, in serum from cancer patients.

When reaction conditions are selected that provide a significant difference in xPKA activities for normal subjects as compared to those afflicted with a carcinoma, the results of the assay may be used to characterize a carcinoma in a subject. That is, the assay may be used to detect or diagnose cancer. In certain embodiments, it may also be used for the determination of a prognosis, determination of drug efficacy, monitoring the status of said subject's response or resistance to a treatment or selection of a treatment for said carcinoma. The carcinoma may be, for example, a carcinoma selected from the group consisting of lung, colon, pancreatic, ovarian, bladder, liver, and prostate cancer.

The methods of the present invention may advantageously be used to characterize a carcinoma or otherwise assess xPKA activity in a variety of subjects. The subject may be, for example, a mammal such as bovine, avian, canine, equine, feline, ovine, porcine, or primate (including humans and non-human primates). A subject may also include mammals of importance due to being endangered, or economic importance, such as animals raised on farms for consumption by humans, or animals of social importance to humans such as animals kept as pets or in zoos. Examples of such animals include but are not limited to: cats, dogs, swine, ruminants or ungulates such as cattle, oxen, sheep, giraffes, deer, goats, bison, camels or horses. In one embodiment, the subject is a human subject. In another embodiment, the subject is bovine, avian, canine, equine, feline, ovine, porcine, or non-human primate.

The subject may have a pre-existing disease or condition, such as cancer. Alternatively, the subject may not have any known pre-existing condition. The subject may also be non-responsive to an existing or past treatment, such as a treatment for cancer.

In general, however, reference values shall be for members of a given species. Thus, for example, xPKA values for human subjects shall only be compared to xPKA values for a statistically significant population of human subjects under equivalent assay conditions. Similarly, xPKA values for non-human subjects shall only be compared to xPKA values for a statistically significant population of non-human subjects of the same species under equivalent assay conditions.

Assay

In general, assays for determination of the activity of extracellular PKA may be carried out by preparing a reaction mixture comprising the sample, a phosphorylation agent, a PKA substrate, and a reagent or system for detecting phosphorylated substrate.

In general, the fluid sample may be derived from any bodily fluid of a subject or subjects. In one embodiment, the sample is previously unfrozen. Exemplary bodily fluids include peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. Additional exemplary bodily fluids include the blastocyl cavity, umbilical cord blood, or maternal circulation which may be of fetal or maternal origin. In one exemplary embodiment, the fluid sample is derived from a bodily fluid selected from among whole blood, sputum, serum, plasma, urine, cerebrospinal fluid, nipple aspirate, saliva, fine needle aspirate, and combinations thereof. In another exemplary embodiment, the fluid sample is derived from a bodily fluid selected from among whole blood, serum, plasma, urine, nipple aspirate, saliva, and combinations thereof. In one preferred embodiment, the fluid sample is derived from blood plasma or serum.

In one embodiment, the sample is treated with a reducing agent or an oxidizing agent. This treatment step may be carried out before the sample is combined with the other components of the reaction mixture, or along with the other components of the reaction mixture.

Reducing agents such as 2-mercaptoethanol, Syringaldazine, sodium hydrosulfite, dithiothreitol, dithioerythreitol, and tris(2-carboxyethyl)phosphine hydrochloride (TCEP) may be used as reducing agents to affect the redox state of the PKA reaction. The reducing agents may preferably be used be at concentrations between 50 μM and 500 mM; in one embodiment, the reducing agents may preferably be used be at concentrations between 50 μM and 100 mM. The reducing agents may be mixed with the sample prior to addition of the sample to the assay, or the reducing agents may be incorporated into the assay mixture. The sample, separately or in the reaction mixture, may be incubated with the reducing agents preferably between 1 minute and 60 minutes.

Oxidizing agents such as diamide, or hydrogen peroxide may be used as oxidizing agents to affect the redox state of the PKA reaction. The oxidizing agents may preferably be used be at concentrations between 5 uM and 100 mM. The reducing agents may be mixed with the sample prior to addition of the sample to the assay, or the reducing agents may be incorporated into the assay mixture. The sample, separately or in the reaction mixture, may be incubated with the reducing agents preferably between 1 minute and 60 minutes.

The phosphorylation agent will typically be ATP although other phosphorylation agents may be employed in certain embodiments.

In general, the PKA substrate may be any peptide substrate for PKA. Exemplary PKA substrates include histone IIa. In a preferred embodiment, the PKA substrate is Kemptide (Leu-Arg-Arg-Ala-Ser-Leu-Gly).

A specific inhibitor of PKA may be useful to discriminate PKA activity from other related kinase activity. One PKA-specific inhibitor which may be used for this purpose is PKI peptide (Ile-Ala-Ala-Gly-Arg-Thr-Gly-Arg-Arg-Gln-Ala-Ile-His-Asp-Ile-Leu-Val-Ala-Ala-OH). Related peptides and shorter peptides derived from the PKI sequence also may be used as PKA-specific inhibitors.

The phosphorylated substrate may be detected using a variety of systems. In general, a probe having affinity for the phosphorylated substrate is conjugated to a “functional group” which is directly or indirectly detectable. The probe may be, for example, an antiphosphoserine antibody. The functional group may be a moiety which is measurable by direct or indirect means (e.g., a radiolabel, a photoactivatable molecule, a chromophore, a fluorophore or a luminophore), or spectroscopic colorimetric labels such as colloidal gold or colored glass or plastic (e.g. polystyrene, polypropylene, latex, etc.) beads. By way of further example, the functional group may be a moiety that is indirectly detectable such as an enzyme (e.g., horse radish peroxidase, alkaline phosphatase etc.), biotin, or a hapten such as digoxigenin. In one exemplary embodiment, an antibody probe is conjugated to a functional group such as a radiolabel, fluorophore, chromophore, chemiluminescent moiety, or enzyme, to facilitate detection. In another embodiment, the probe is conjugated to one member of an affinity pair, e.g., biotin, and a detectable label is conjugated to the second member of the affinity pair, e.g., avidin or streptavidin.

Exemplary radiolabels include ³H, ¹²⁵I, ³⁵S, ¹⁴C, ³²_{P}, and ³³P.

Exemplary chromophore/luminophores include any organic or inorganic dyes, fluorophores, phosphophores, light absorbing nanoparticles (e.g., Au, Ag, Pt, Pd), combinations thereof, or the metalated complexes thereof.

Exemplary organic dyes are selected from the group consisting of coumarins, pyrene, cyanines, benzenes, N-methylcarbazole, erythrosin B, N-acetyl-L-tryptophanamide, 2,5-diphenyloxazole, rubrene, and N-(3-sulfopropyl)acridinium. Specific examples of preferred coumarins include 7-aminocoumarin, 7-dialkylamino coumarin, and coumarin 153. Exemplary benzenes include 1,4-bis(5-phenyloxazol-2-yl)benzene and 1,4-diphenylbenzene. Exemplary cyanines include oxacyanines, thiacyanines, indocyanins, merocyanines, and carbocyanines. Other exemplary cyanines include ECL Plus, ECF, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, CypHer5, Dye-33, Cy7, Cy5, Cy5.5, Cy3Cy5 ET, Cy3B, Cy3, Cy3.5, Cy2, CBQCA, NIR1, NIR2, NIR3, NIR4, NIR820, SNIR1, SNIR2, SNIR4, Merocyanine 540, Pinacyanol-Iodide, 1,1-Diethyl-4,4-carbocyanine iodide, Stains All, Dye-1041, or Dye-304.

Exemplary inorganic dyes include metalated and non-metalated porphyrins, phthalocyanines, chlorins (e.g., chlorophyll A and B), and metalated chromophores. Exemplary porphyrins include porphyrins selected from the group consisting of tetra carboxy-phenyl-porphyrin (TCPP) and Zn-TCPP. Exemplary metalated chromophores include ruthenium polypyridyl complexes, osmium polypyridyl complexes, rhodium polypyridyl complexes, 3-(1-methylbenzoimidazol-2-yl)-7-(diethylamino)-coumarin complexes of iridium(III), and 3-(benzothiazol-2-yl)-7-(diethylamino)-coumarin complexes with iridium(III).

Exemplary fluorophores and phosphophores include phosphorescent dyes, fluoresceines, rhodamines (e.g., rhodamine B, rhodamine 6G), and anthracenes (e.g., 9-cyanoanthracene, 9,10-diphenylanthracene, 1-Chloro-9,10-bis(phenylethynyl)anthracene).

Having described the invention in detail, it will be apparent that modifications and variations are possible without departing from the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes, such as changing the reaction pH to affect the ORP, can be made in the specific embodiments that are disclosed and still obtain a like or similar result without departing from the spirit and scope of the invention.

### Example 1

Serum samples from patients with cancer and individuals apparently without cancer were obtained from ProMedDx, LLC and from ProteoGenex. In one embodiment blood samples from prostate cancer patients and normal controls presumably without cancer were assayed for apparent xPKA activity. In this assay extracellular PKA in samples was mixed with a defined peptide used as a substrate. The substrate peptide was bound to the wells of the microtiter assay plate. Phosphorylation of the peptide was detected using biotinylated phosphoserine antibody, which was in turn was detected in an ELISA format using peroxidase-conjugated to streptavidin. Detection of the bound peroxidase was established using a color-producing peroxidase substrate included in the assay kit. Bovine PKA catalytic unit was used at varying concentrations to develop a standard activity curve. The detail of the assay protocol is described below.

1. Reference: Kit Instructions

2. Materials
a. MESACUP Protein Kinase Assay Kit (MBL Code No. 5230)
b. ATP: 10 mM in water
   i. Dissolve 60 mg ATP (Sigma Prod. No. A2383) in 1.0 ml water
   ii. Determine the absorbance of a 1/1000 dilution in PBS at 259 nm
   iii. Store at −20° C.
   iv. Immediately before use dilute to 10 mM based on the absorbance and the molar extinction coefficient (E₂₅₉, pH 7=15,400)
c. PKI inhibitor: 0.5 mM in water (Santa Cruz Prod. No. sc-201160)
   i. Dissolve 1 mg in 1.0 ml water
   ii. Store at −20° C.
d. PKA diluent: 25 mM KH₂PO₄, 5 mM EDTA, 150 mM NaCl, 50% (w/v) glycerol, 1 mg/ml BSA, and various concentrations of reductant or oxidant (2-mercaptoethanol, dithiothreitol, dithioerythritol, or diamide) as indicated directly in the data figures by concentration or by oxidation-reduction potential, pH 6.5
e. PKA catalytic subunit standard:
   i. Dissolve bovine PKA (Sigma Prod. No. P2645) in cold PKA diluent to a final concentration of 1 g/ml
   ii. Store at −20° C.
f. Peroxidase substrate solution (Sigma Prod. No. T8665)

3. Procedure
g. Prepare samples
   i. Thaw serum samples
   ii. Centrifuge 5 minutes at 16,000×g
   iii. Collect the clear supernatant
   iv. Mix 0.0108 ml supernatant with 0.0012 ml diluent in a dilution plate, two wells per sample
   v. Incubate one hour at room temperature
h. Prepare calibration curve
   i. Prepare serial ½ dilutions of PKA 20-0.4 ng/ml in PKA diluent
   ii. Dispense 0.012 ml per well of a dilution plate
i. Prepare reaction buffer to final concentrations of:
   i. 25 mM tris-HCl, pH 7.0
   ii. 3 mM MgCl₂
   iii. 1 mM ATP
   iv. 0 or 5 uM PKI
j. Add 0.108 ml reaction buffer (with or without PKI) to each sample or calibrator well of the dilution plate
k. Transfer 0.100 ml per well to assay plate
l. Incubate 20 minutes at 25° C. with shaking at 750 rpm
m. Add 0.100 ml kit stop solution per well
n. Wash three times with kit wash buffer
o. Add 0.100 ml kit biotinylated anti-phosphoserine per well
p. Incubate 60 minutes at 25° C. with shaking at 750 rpm
q. Wash three times with kit wash buffer
r. Add 0.100 ml kit peroxidase-conjugated streptavidin per well
s. Incubate 60 minutes at 25° C. with shaking at 750 rpm
t. Wash three times with kit wash buffer
u. Add 0.100 ml peroxidase substrate solution per well
v. Incubate 3 minutes at 25° C. with shaking at 750 rpm
w. Add 0.100 ml kit stop solution per well
x. Shake briefly until well mixed
y. Read absorbance at 450 nm

4. Calculation of results
z. Plot absorbance versus concentration of the calibration curve
aa. Perform a least squares linear regression on the data or use a third order polynomial curve (cubic spline) on the data to determine a standard curve
bb. Determine kinase concentration in the samples from interpolation of the values on the standard curve
cc. Calculate net PKA in the samples
   i. Net PKA (ng/ml)=Kinase (0 uM PKI)−Kinase (0.5 uM PKI).

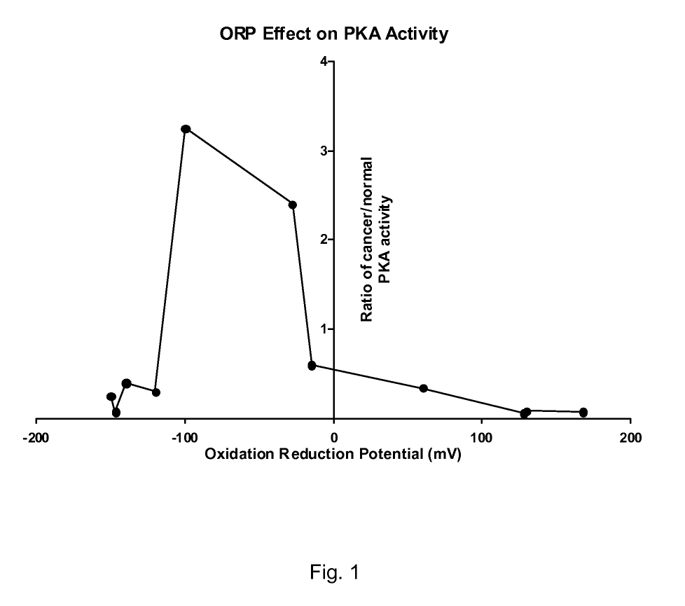

The oxidation-reduction potential (ORP) of the sample preparation buffers was measured using a platinum redox probe. ORPs are expressed in mV. The apparent PKA activity for samples from cancer patients relative to normal samples were plotted for various ORP value solutions. As shown in FIG. 1, under oxidizing conditions, mild reducing conditions or highly reducing conditions the apparent xPKA activity in serum samples from prostate cancer patients was lower than that from samples from individuals apparently without cancer. Under moderate reducing conditions, the apparent xPKA activity in serum samples from prostate cancer patients was higher than that from samples from individuals apparently without cancer.

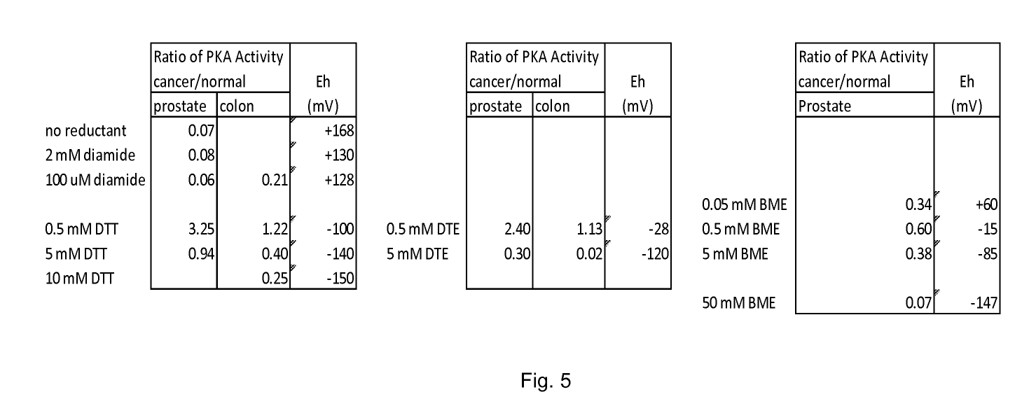

FIG. 5 shows the relative apparent xPKA activities in samples from prostate and colon cancer patients relative to those from individuals apparently without cancer with various concentrations of oxidant or reductant used in the sample preparation buffer. The same pattern of xPKA activity is observed with both the prostate and the colon cancer patient samples. The relative PKA activities of cancer patients is higher, lower, or the same as that of individuals apparently without cancer, depending upon the concentration of oxidant or reductant used in the sample preparation buffer.

### Example 2

xPKA activities were determined using the procedure described in Example 1 with the exception that oxidant addition, reductant addition, or no addition was made to the reaction buffer. Samples were not preincubated in sample buffer, but were incubated for 5 minutes at 25 C in reaction buffer with shaking at 750 rpm prior to adding the reaction mixtures to the assay plate wells.

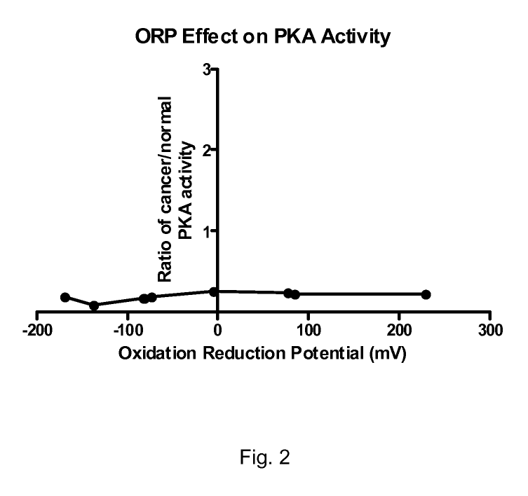

The oxidation-reduction potential (ORP) of the reaction buffers was measured using a platinum redox probe. ORPs are expressed in mV. The apparent PKA activity for samples from cancer patients relative to normal samples were plotted for various ORP value solutions. With a shorter treatment with oxidant or reductant (FIG. 2) there is lower overall activity observed in the samples and the level of apparent PKA activity of the cancer patient samples relative to those from normals is consistently low (below 0.4:1)

### Example 3

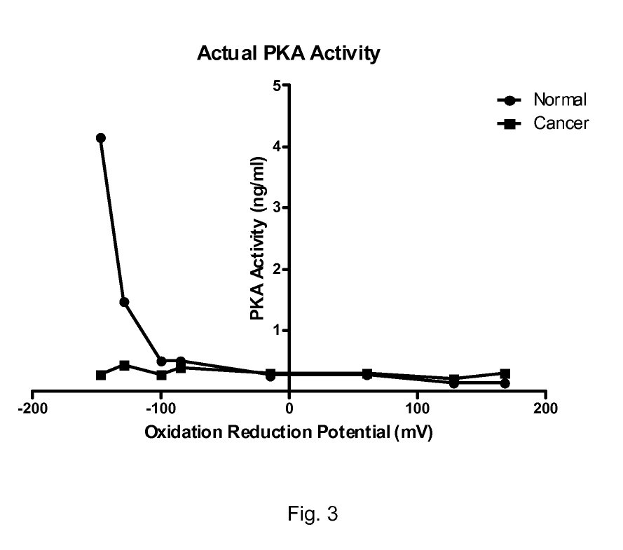

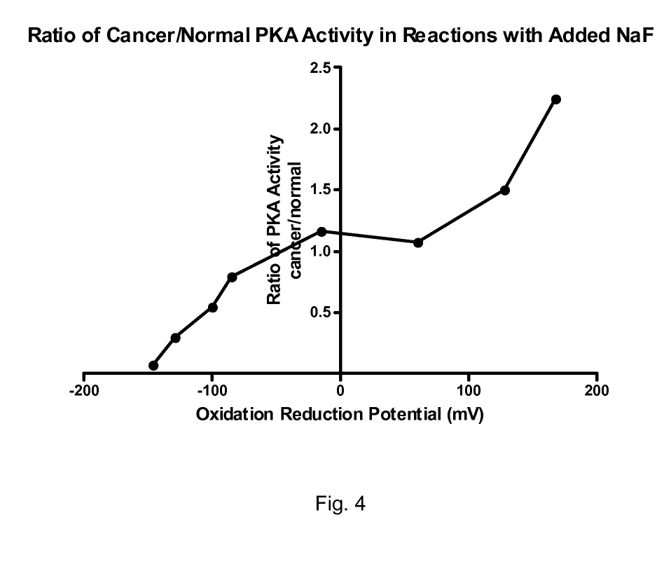

Samples were treated as in example 1. In one set of reaction mixes NaF was added at a concentration of 2 mM. NaF is a nonspecific inhibitor of phosphatases. A reaction run with NaF inhibitor provides a measure of actual xPKA activity. FIG. 3 shows that with phosphatase inhibition, the actual xPKA activity in samples from cancer patients varied little with changes in redox conditions. In normal subjects with phosphatase inhibition however, actual xPKA activity was reduced by about 50% overall and the xPKA was still subject to regulation by redox conditions. This demonstrates the complex interplay of enzyme activities and redox conditions that control apparent xPKA activity and that controls the relationship between apparent xPKA activity levels in cancer patients and those individuals apparently without cancer. FIG. 4 shows the ratio of apparent xPKA activity (cancer subjects/normal subjects) as a function of oxidation reduction potential in reactions containing NaF. The ratio of cancer/normal xPKA activity varies dramatically depending upon redox conditions.

### CITATION LIST

1. PEARCE, L. R., D. KOMANDER, AND D. R. ALESSI, THE NUTS AND BOLTS OF AGC PROTEIN KINASES. NAT REV MOL CELL BIOL, 2010. 11(1): P. 9-22.
2. WALSH, D. A., ET AL., THE INHIBITOR PROTEIN OF THE CAMP-DEPENDENT PROTEIN KINASE, IN PEPTIDES AND PROTEIN PHOSPHORYLATION, B. E. KEMP, EDITOR. 1990, CRC PRESS, INC.: BOCA RATON, Fla. P. 43-84.
3. CHO, Y. S., Y. N. LEE, AND Y. S. CHO-CHUNG, BIOCHEMICAL CHARACTERIZATION OF EXTRACELLULAR CAMP-DEPENDENT PROTEIN KINASE AS A TUMOR MARKER. BIOCHEM BIOPHYS RES COMMUN, 2000. 278(3): P. 679-84.
4. CHO, Y. S., ET AL., EXTRACELLULAR PROTEIN KINASE A AS A CANCER BIOMARKER: ITS EXPRESSION BY TUMOR CELLS AND REVERSAL BY A MYRISTATE-LACKING CALPHA AND RIIBETA SUBUNIT OVEREXPRESSION. PROC NATL ACAD SCI U S A, 2000. 97(2): P. 835-40.
5. NESTEROVA, M. V., ET AL., AUTOANTIBODY CANCER BIOMARKER: EXTRACELLULAR PROTEIN KINASE A. CANCER RES, 2006. 66(18): P. 8971-4.
6. HUMPHRIES, K. M., C. JULIANO, AND S. S. TAYLOR, REGULATION OF CAMP-DEPENDENT PROTEIN KINASE ACTIVITY BY GLUTATHIONYLATION. J BIOL CHEM, 2002. 277(45): P. 43505-11.
7. ROLLINS, G., PSA TESTING: YES, NO, MAYBE. CLIN LAB NEWS, 2009. 35(6): P. 1, 3-5.
8. SMITH, D. S., P. A. HUMPHREY, AND W. J. CATALONA, THE EARLY DETECTION OF PROSTATE CARCINOMA WITH PROSTATE SPECIFIC ANTIGEN: THE WASHINGTON UNIVERSITY EXPERIENCE. CANCER, 1997. 80(9): P. 1852-6.
9. SCHRODER, F. H., ET AL., SCREENING AND PROSTATE-CANCER MORTALITY IN A RANDOMIZED EUROPEAN STUDY. N ENGL J MED, 2009. 360(13): P. 1320-8.
10. JEMAL, A., ET AL., CANCER STATISTICS, 2008. CA CANCER J CLIN, 2008. 58(2): P. 71-96.
11. AHMED, H. U., ET AL., IS IT TIME TO CONSIDER A ROLE FOR MRI BEFORE PROSTATE BIOPSY? NAT REV CLIN ONCOL, 2009. 6(4): P. 197-206.
12. ELMORE, J. G., ET AL., SCREENING FOR BREAST CANCER. JAMA, 2005. 293(10): P. 1245-1256.
13. HARRIS, L., ET AL., AMERICAN SOCIETY OF CLINICAL ONCOLOGY 2007 UPDATE OF RECOMMENDATIONS FOR THE USE OF TUMOR MARKERS IN BREAST CANCER. J CLIN ONCOL, 2007. 25(33): P. 5287-312.
14. JEMAL, A., ET AL., CANCER STATISTICS, 2009. CA CANCER J CLIN, 2009: P. CAAC 20006.
15. BOUDREAU, A. C., ET AL., SIGNALING PATHWAY ADAPTATIONS AND NOVEL PROTEIN KINASE A SUBSTRATES RELATED TO BEHAVIORAL SENSITIZATION TO COCAINE. J NEUROCHEM, 2009. 110(1): P. 363-77.
16. SMITH, B. D., ET AL., FUTURE OF CANCER INCIDENCE IN THE UNITED STATES: BURDENS UPON AN AGING, CHANGING NATION. J CLIN ONCOL, 2009. 27(17): P. 2758-65.
17. AMERICAN CANCER SOCIETY. CANCER FACTS & FIGURES 2008. 2008, AMERICAN CANCER SOCIETY: ATLANTA.

1. A method for characterizing a carcinoma in a subject, the method comprising the steps of
assaying a sample of a previously unfrozen bodily fluid derived from the subject for extracellular PKA activity, the assay comprising preparing a reaction mixture comprising the previously unfrozen sample, a PKA peptide substrate, a phosphorylation agent, incubating the prepared mixture, and detecting phosphorylated substrate formed in the incubated mixture, and
comparing the amount of phosphorylated substrate formed in the assay with a reference value, the reference value being the amount of phosphorylated substrate formed in a mixture under equivalent redox conditions for a sample of bodily fluid derived from a population of normal subjects of the same species.

2. The method of claim 1 wherein extracellular PKA derived from a statistically significant population of subjects unafflicted with a carcinoma and extracellular PKA derived from a statistically significant population of subjects afflicted with a carcinoma have significantly different activities for the phosphorylation of the PKA substrate under the assay conditions.

3. The method of claim 2 wherein a ratio of the activity of extracellular PKA derived from the population of subjects unafflicted with a carcinoma to the activity of extracellular PKA derived from the population of subjects afflicted with a carcinoma for the phosphorylation of the PKA substrate is at least about 1.5:1 or less than 0.7:1, respectively.

4. The method of claim 2 wherein a ratio of the activity of extracellular PKA derived from the population of subjects unafflicted with a carcinoma to the activity of extracellular PKA derived from the population of subjects afflicted with a carcinoma for the phosphorylation of the PKA substrate is at least about 2:1 or less than 0.5:1, respectively.

5. The method of claim 2 wherein a ratio of the activity of extracellular PKA derived from the population of subjects unafflicted with a carcinoma to the activity of extracellular PKA derived from the population of subjects afflicted with a carcinoma for the phosphorylation of the PKA substrate is at least about 3:1 or less than 0.2:1, respectively.

6. The method of claim 1, wherein preparing the reaction mixture comprises treating the sample with a reductant selected from the group consisting of 2-mercaptoethanol, dithioerythritol and dithioerythritol at a concentration between 5 μM and 500 mM.

7. The method of claim 1, wherein preparing the reaction mixture comprises treating the sample with an oxidizing agent.

8. The method of claim 7 wherein the oxidizing agent is a diamide at a concentration between 5 μM and 50 mM or hydrogen peroxide at a concentration between 1 μM and 500 mM.

9. The method of claim 1, wherein phosphorylated substrate formed in the incubated mixture is detected by a method not requiring the use of radioactive elements.

10. A method for characterizing a carcinoma in a subject, the method comprising the steps of
incubating a mixture comprising a sample of a bodily fluid derived from the subject, a PKA peptide substrate, a phosphorylation agent, and a reducing agent, the mixture having an oxidation reduction potential value that is less than −150 mV or greater than −20 mV, and
detecting phosphorylated substrate formed in the incubated mixture.

11. The method of claim 10, wherein the carcinoma is selected from the group consisting of lung, colon, pancreatic, ovarian, bladder, liver, and prostate cancer.

12. The method of claim 10, wherein characterizing comprises a detection, diagnosis, prognosis, determination of drug efficacy, monitoring the status of said subject's response or resistance to a treatment or selection of a treatment for said carcinoma.

13. A method for determining the activity of extracellular cyclic AMP dependent protein kinase A (PKA) in a sample derived from a bodily fluid and previously unfrozen for the phosphorylation of the PKA substrate, the method comprising the steps of
incubating a mixture comprising the sample, a PKA peptide substrate, a phosphorylation agent, and a reducing agent, the mixture having an oxidation reduction potential value that is less than −110 mV and greater than −20 mV, and
detecting phosphorylated substrate formed in the incubated mixture.

14. A method for determining the activity of extracellular cyclic AMP dependent protein kinase A (PKA) in a sample derived from a bodily fluid and previously unfrozen for the phosphorylation of the PKA substrate, the method comprising the steps of
incubating a mixture comprising the sample, a PKA peptide substrate, a phosphorylation agent, and a reducing agent, the mixture having an oxidation reduction potential value that is between −20 mV and 200 mV, and
detecting phosphorylated substrate formed in the incubated mixture.

15. The method of claim 14, wherein the bodily fluid is peripheral blood, serum, plasma, ascites, urine, cerebrospinal fluid (CSF), sputum, saliva, bone marrow, synovial fluid, aqueous humor, cerumen, broncheoalveolar lavage fluid, semen, prostatic fluid, cowper's fluid or pre-ejaculatory fluid, sweat, fecal matter, tears, cyst fluid, pleural and peritoneal fluid, lymph, chyme, chyle, bile, intestinal fluid, pus, sebum, vomit, mucosal secretion, stool water, pancreatic juice, lavage fluids from sinus cavities, or bronchopulmonary aspirates.

16. The method of claim 1, wherein the bodily fluid is serum.

17. The method of claim 10, wherein the reducing agent is selected from the group consisting of 2-mercaptoethanol, dithiothreitol, and dithioerythritol and the concentration of the reducing agent in the mixture is at least 0.005 mM.

18. The method of claim 1, wherein the phosphorylation agent is ATP.

19. The method of claim 1, wherein the PKA peptide substrate is Kemptide.

20. A kit for the assay of PKA activity comprising a PKA substrate, a phosphorylation agent, a control PKA of known activity and an oxidizing agent or a phosphatase inhibitor.
